(19) United States
(12) Patent Application Publication
(10) Pub. No.: US 2022/0100196 A1
(43) Pub. Date: Mar. 31, 2022

(54) Smart Thermal Tracking to Guide Surface Sanitization

(71) Applicant: Duke University, Durham, NC (US)

(72) Inventors: Weston ROSS, Durham, NC (US); Patrick CODD, Durham, NC (US); Daniel BUCKLAND, Durham, NC (US); Matthew TUCKER, Durham, NC (US); Guangshen MA, Durham, NC (US)

(21) Appl. No.: 17/138,628

(22) Filed: Dec. 30, 2020

Related U.S. Application Data
(60) Provisional application No. 63/083,451, filed on Sep. 25, 2020.

(51) Int. Cl. G05D 1/02; G06T 19/00; G06T 7/00; G06T 17/00; G05D 1/00; G01J 5/00; A47L 11/40; A61L 2/24; A61L 2/22; A61L 2/10

(52) U.S. Cl. CPC G05D 1/0219; H04N 13/20; G06T 7/0002; G06T 17/00; G05D 1/0251; G05D 1/0038; G05D 1/0044; G01J 5/0066; A47L 11/4011; A47L 11/4061; A61L 2/24; A61L 2/22; A61L 2/10; G06T 2207/10048; G06T 19/006

(57) ABSTRACT

The present disclosure describes a system and method for using thermal cameras to detect surfaces that have been in contact with individuals. The detection can be used to guide focused cleaning of the surfaces.

### CROSS-REFERENCE TO RELATED APPLICATIONS

This application claims the benefit of U.S. Provisional Application No. 63/083,451, filed Sep. 25, 2020, for “Smart Thermal Tracking to Guide Surface Sanitization,” which is incorporated herein by reference.

### BACKGROUND

Infections associated with health care have been a long-standing problem. These infections contribute to patient morbidity and mortality as well as health care costs, and therefore cleaning practices play a central role in many hospital policies. Additionally, the COVID-19 pandemic has heightened the concern for virus spread through touching communal surfaces. Rigorous cleaning practices have extended to virtually all public and commercial spaces in attempts to slow the spread of the virus. However, these practices come at a cost.

In the example of hospitals, it is not currently possible to discern exactly which surfaces have been touched, and so it is a common sanitization practice to fully clean all surfaces of a room that has been occupied by at-risk patients. This is an inefficient process that not only consumes human resources but also removes the room from service for lengthy periods of time. Hence, there is a need for improved cleaning efficiency while maintaining high quality standards.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a method of identifying a region of a surface that has been touched by a subject, the method comprising, consisting of, or consisting essentially of: monitoring, using a thermal imaging device, an area of interest comprising the surface; detecting, using an image processing system, heat that is transferred to or from the region of the surface after being contacted by the subject; and storing an indication of the contacted region of the surface.

In some embodiments, the method further comprises reporting the contacted region, which may include creating a visual representation of the area of interest upon which a graphical overlay of the contacted region is displayed. The visual representation may be a virtual or augmented reality image in certain configurations.

The method may further include directing a cleaner to the contacted region. The cleaner may include a cleaning robot, and directing may include navigating the cleaning robot to the contacted region.

The method may also include tracking a number of times that one or more regions of at least one surface of the area of interest have been contacted; and reporting at least one of the one or more regions that have been contacted most frequently for more intensive or frequent cleaning.

In some embodiments, detecting transferred heat may include detecting, using the image processing system, a change in temperature of the region of the surface; determining whether the change in temperature is consistent with human contact with the region of the surface; and if the change in temperature is consistent with a human contact, identifying the region as a contacted region. Determining whether the change in temperature is consistent with human contact may include evaluating the change temperature based on at least one of a magnitude or rate of change of change in temperature of the region of the surface.

In some embodiments, the method may include, prior to monitoring, generating a three-dimensional representation of one or more surfaces in the area of interest.

Another aspect of the present disclosure provides a system comprising, consisting of, or consisting essentially of: a thermal imaging device configured to capture an image of an area of interest that has at least one surface; an image processing system configured to detect heat transferred to or from a region of the at least one surface after the region has been contacted by a subject, and a storage device configured to store an indication of the contacted region of the surface.

The system may further include a communication interface configured to report the contacted region. In some embodiments, the system may include a camera configured to obtain a visual representation of the area of interest; and a display device configured to display a graphical overlay corresponding to the contacted region on the visual representation. The visual representation may be a virtual or augmented reality image in various embodiments. The communication interface may be further configured to direct a cleaner to the contacted region. The cleaner may include, for example, a cleaning robot.

In some embodiments, the system further includes: a processor configured to track a number of times that one or more regions of at least one surface of the area of interest have been contacted; and a communication interface configured to report at least one of the one or more regions that have been contacted most frequently for more intensive or frequent cleaning.

The image processing system, in one configuration, may be configured to: detect a change in temperature of the region of the surface; determine whether the change in temperature is consistent with human contact with the region of the surface; and if the change in temperature is consistent with a human contact, identify the region as a contacted region. The image processing system may be configured to determine whether the change in temperature is consistent with human contact by evaluating the change temperature based on at least one of a magnitude or rate of change of change in temperature of the region of the surface.

In certain embodiments, the system may include a three-dimensional modeling system configured to generate a three-dimensional model of one or more surfaces in the area of interest. The three-dimensional modeling system may include a three-dimensional imager.

The system may include a plurality of thermal imaging devices configured to work in concert with one another and with the image processing system to monitor the area of interest.

Yet another aspect may include a non-transitory computer-readable medium comprising program code that, when executed a processor, causes the processor to perform a method of identifying a region of a surface that has been contacted by a subject, the method comprising, consisting of, or consisting essentially of: monitoring, using a thermal imaging device, an area of interest comprising the surface; detecting, using an image processing system, heat that is transferred to or from the region of the surface after being contacted by the subject; and storing an indication of the contacted region of the surface.

These and other aspects will be described more fully with reference to the Figures and Examples disclosed herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 is a schematic diagram of touch detection system.

FIG. 2 depicts thermal signatures captured over time.

FIG. 3 illustrates the capture of multiple thermal signatures to create a heat map.

FIG. 4A illustrates the capture of multiple thermal signatures to create a heat map.

FIG. 4B is a visual representation including graphical overlays corresponding to touched regions.

FIG. 5 is a schematic diagram of a 3D modeling system for capturing a 3D model of an area of interest.

FIG. 6 is a visual representation of an area of interest with graphical overlays representing touched regions.

FIG. 7 illustrates a cleaning robot being navigated to a touched region.

FIG. 8 illustrates an embodiment including multiple thermal imaging cameras that work in concert.

FIG. 9 is a flowchart of a method for touch detection.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skill in the art to which this disclosure belongs.

The COVID-19 pandemic has spurred expanded sanitation regimes in communal spaces, whereby any potentially touchable surfaces are repetitively cleaned to reduce the risk of transmission of infection. This is occurring in many clinical, commercial, educational, and public settings, but especially so in high-risk areas such as Emergency Department Triage. For example, current triage practice in the Duke University Hospital Emergency Department involves interviewing a patient on first arrival in one of four triage rooms, and then doing a full cleaning of all the room's surfaces if it is determined that the patient is at a reasonable risk for having a coronavirus infection. This is an inefficient process that takes the triage room out of use for up to 45 minutes while it is being cleaned, as it must be assumed that the patient could have touched any of the surfaces in the room. Having a method to identify high-risk surfaces for targeted sterilization would improve sanitation and manage risk. The present disclosure addresses these and other challenges by providing a solution that can reduce the time of cleaning. This is accomplished by directing the cleaning to only surfaces that have been touched, thus improving efficiency, reducing operating costs, and increasing patient throughput by ensuring that triage rooms are available to be used as much as possible.

Although the systems and methods disclosed herein are generally described with reference to sanitization of clinical surfaces, the systems and methods can be equally applied to varying degrees of cleaning (e.g., cleaning, disinfecting, sanitizing, sterilizing, etc.) and can be used in a variety of settings (e.g., workplaces, lobbies, gyms, salons, restaurants, public transportation, classrooms, laboratories, airports, grocery stores, retail space, childcare environments, in homes, etc.). Nor is the present disclosure intended only for elimination of viruses, bacteria, etc. It also lends itself to a wide variety of uses outside medical applications, such as in security monitoring, manufacturing compliance, clean rooms, or any other situation where it is useful to know if and/or where a surface has been touched.

As used herein, the term “touch” is not limited to describe contacting a surface through a hand or finger, but can also include contact with any portion of a subject's body in such a way as to confer heat. In addition, a human can transfer heat to a surface without physical contact, such as breathing or coughing on the surface or otherwise transferring bodily fluids to the surface. Therefore, “contact” is intended to be construed broadly to include both direct physical contact and indirect forms of contact in which a transfer of heat is capable of being detected. Although the present disclosure will frequently refer to “touch” or “touch detection,” those of skill in the art will recognize that the terms could be interchangeably replaced with “contact” or “contact detection.”

In the case of a surface being colder that the subject, the subject may transfer heat to the surface, temporarily increasing the temperature of the surface. However, when the surface is warmer, the subject may receive heat from the surface, temporarily decreasing the temperature of the surface. Thus, the present disclosure relates to changes of heat as a result of the proximity of a human body to a surface, which may indicate that disease causing agents have been transferred to the surface as a result of contact with the body or bodily emissions.

One aspect of the present disclosure provides a system to determine surfaces that have been touched, an application of which is to guide surface sanitization. The system comprises a thermal imaging device and an image processing system. In some embodiments, the thermal imaging device is a thermal camera. The thermal imaging device is configured to monitor the space of interest and particularly any surfaces of interest. Some non-limiting examples of surfaces of interest include items intended to be touched, such as handrails, doorknobs, faucet handles, examination tables, etc., as well as incidental surfaces such as chairs, counters, windows, walls, etc.

The image processing system is configured to analyze the image produced by the thermal imaging device and to discern when a surface has been touched. Each time a surface is touched by anyone in the space (e.g., patient, care provider, etc.), that surface may be logged as “contacted.” The log can then be provided to the appropriate personnel to direct cleaning of those surfaces or to third parties to avoid areas of possible contamination. The log may also contain more detailed information such as where on the surface the contact occurred, for how long the contact was made, the number of times the surface was contacted, etc.

In some embodiments, the system includes an optional display device to report the results of the log. The report can be provided in any suitable format and include any representation of the data. Some example formats include a visual representation of the space overlaid with graphical markers, a descriptive listing, representative icons, projection to screens, handheld devices, printed report, images, augmented reality projections, virtual reality projections, and/or any other format for communicating which surfaces have been touched. In a non-limiting example, the display can include a graphical user interface that is implemented on a touch-screen system with an IP54-certified casing suitable for hospital sterilization procedures. Similarly, the display could include a mobile communication device, such as a cellular telephone, or a virtual or augmented reality headset.

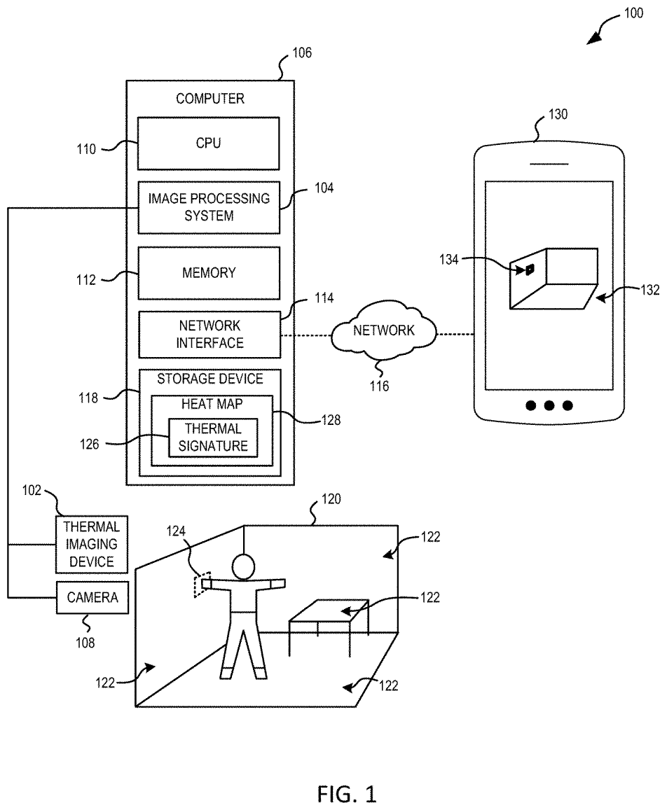

FIG. 1 is a schematic diagram of a touch detection system 100 according to one embodiment of the present disclosure. The touch detection system 100 may include a thermal imaging device 102, which may be implemented, for example, by a T-series® high-performance thermal camera available from FLIR systems of Wilsonville, Oreg. In some embodiments, the thermal imaging device 102 may include, or be coupled to, a wide-angle lens to maximize coverage of a particular area.

The touch detection system 100 may further include an image processing system 104 for processing and aggregating data received from the thermal imaging device 102. In various embodiments, the image processing system 104 may be a component of the thermal imaging device 102, a standalone component, or, as illustrated, a hardware and/or software component of a computer 106. As described more fully below, the image processing system 104 may be used to aggregate multiple thermal images acquired at different times and/or from different thermal imaging devices 102. The image processing system 104 may be configured to perform distortion compensation, image fusion, object recognition/tracking, and a variety of other functions using standard techniques to implement the processes and features described herein.

The image processing system 104 may be connected via a wired or wireless connection to the thermal imaging device 102. Optionally, the image processing system 104 may be coupled to a visible light camera 108 (or, simply, “camera”), including, without limitation, a digital image sensor that captures light in the visible spectrum. Although the thermal imaging device 102 and visible light camera 108 are illustrated as separate components, those skilled in the art may will recognize that the devices may be combined in various embodiments. For example, a combined thermal imaging device 102 and visible light camera 108 may implement FLIR MSX® (Multi-Spectral Dynamic Imaging), which adds visible light details to thermal images in real time for greater clarity, as well as embedding edge and outline detail onto thermal readings.

The computer 106 may be controlled by a central processing unit (CPU) 110, such as a microprocessor, although other types of controllers or processing devices may be used, such as a microcontroller, digital signal processor (DSP), application specific integrated circuit (ASIC), field-programmable gate array (FPGA) or like device. The CPU 110 may execute instructions stored in a memory 112, which may be implemented using any suitable non-transitory computer-readable medium, such as random access memory (RAM), read-only memory (ROM), electronically erasable programmable read-only memory (EEPROM), or the like.

The computer 106 may further include a network interface 114 for connecting the computer 106 to a network 116, such as a local area network (LAN) and/or wide area network (WAN), including the Internet. The network interface 114 may implement any suitable network protocols using a wireless or wired transmission medium.

The computer 106 may also include a storage device 118, such as a hard disk drive (HDD), solid-state drive (SSD), and/or or optical storage unit, for long-term storage of data and/or application programs. The storage device 118 may be local to the computer 106, as shown, or could be implemented remotely in the cloud. Furthermore, the various components described above may be implemented in separate devices, local or remote, that work in concert to perform the operations disclosed herein.

FIG. 1 also illustrates an area of interest 120 including a plurality of surfaces 122. The area of interest 120 could be, without limitation, an emergency department (ED) triage room, intensive care unit (ICU), operating room (OR), classroom, hallway, office space, conference room, airport terminal, patient room, hallway, waiting area, or the like. The surfaces 122 could be, without limitation, walls, doors, tables, counters, fixtures, medical equipment, or the like.

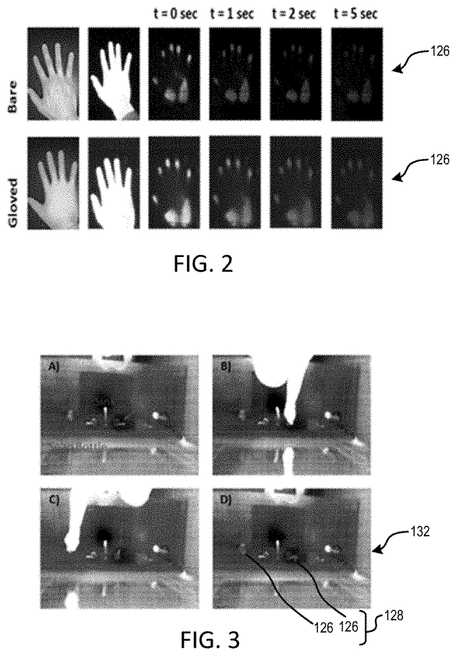

In operation, when a subject touches a region 124 of a surface 122, a thermal signature 126 on the touched surface 122 is detectable for several seconds after contact. Referring also to FIG. 2, various thermal signatures 126, as captured by a thermal imaging device 102, are illustrated for five seconds after being touched with a bare hand and a gloved hand. Captured thermal signatures 126, such as those shown in FIG. 2, may be aggregated and analyzed by the image processing system 104 to generate a cumulative heat map 128 of surfaces 122 and/or regions 124 touched.

Digital representations of the heat map 128 and/or thermal signatures 126 may be stored in the memory 112 and/or storage device 118 using any suitable data structure and may include, without limitation, coordinates, temperature readings, time/date stamps, thermal photographs, visible light photographs, or the like.

In some embodiments, the image processing system 104 may determine whether a change in temperature detected by the thermal imaging device 102 is consistent with human contact. Not all transient thermal signatures 126 are indicative of human contact. For example, turning on or off certain equipment may generate a transient change in temperature.

The image processing system 104 may be configured to filter possible thermal signatures 126 using a variety of techniques in order to store only thermal signatures that are consistent with human contact. For example, the image processing system 104 may filter out certain thermal signatures 126 based on an overall magnitude of the detected temperature, such as where the thermal signature is too warm or too cold to be consistent with human contact.

Likewise, as illustrated in FIG. 2, thermal signatures consistent with human contact typically decay with a predictable rate of change. This may differ, for example, from a medical device that is turned on or off, which may exhibit a constant temperature or a different rate of change of temperature. If the image processing system 104 determines that a change in temperature is consistent with human contact, a record of it may be stored. Otherwise, it may be discarded/filtered.

In some embodiments, the image processing system 104 may use more other techniques to determine whether a thermal signature 126 is of human origin. For instance, the image processing system 104 may employ image recognition to determine that a thermal signature 126 is in the shape of a human hand, as shown in FIG. 2. Alternatively, or in addition, the image processing system 104 may employ object tracking to track, for example, a subject's hand and note that the thermal signature 126 was created by contact between the subject's hand and the surface. Object recognition and tracking are known in the art and rely on a variety of techniques, including convolutional neural networks (CNNs).

In one embodiment, the touch detection system 100 is capable of determining and reporting regions 124 and/or surfaces 122 that are touched, as well as regions 124 and/or surfaces 122 that are high-touch or “hot-spot” areas. The identification of one or more of these high-touch areas can then be used for further actions, such as targeting the areas for rigorous cleaning or providing them with disposable covers.

Referring again to FIG. 1, the touch detection system 100 may include, or be accessible to, a display device 130, which may be embodied as a cellular telephone (as shown) or other suitable display device, including, without limitation, a tablet, computer monitor, television, virtual reality (VR) headset, augmented reality (AR) headset, or the like.

The display device 130, by itself or in conjunction with the computer 106, may be used to create a visual representation 132 of the area of interest 120. In some embodiments, a graphical overlay 134 corresponding to one or more of the contacted surface 122, contacted region 124, thermal signatures 126 and/or heat map 128 is/are displayed upon the visual representation 132.

Users of the display device 130 may include a cleaner tasked with cleaning the area of interest 120, as well individuals who wish to avoid contacted regions 124 in the area of interest 120, such as hospital visitors, staff, students, passengers, etc. In some embodiments, visitors to a facility may be able to access stored heat maps 128 in order to navigate an area without touching a possibly infected surface 122.

FIG. 3 includes overhead images of a subject using a sink, as captured by a thermal imaging device 102. Image A) identifies the parts of a sink as seen from overhead. Image B) is a thermal image of the subject touching a sink handle. Image C) shows the subject then touching a soap bottle. Image D) shows a processed visual representation 132 generated by the image processing system 104 that highlights the detected thermal signatures 126 resulting from human contact. The aggregated thermal signatures 126 may be referred to as a heat map 128.

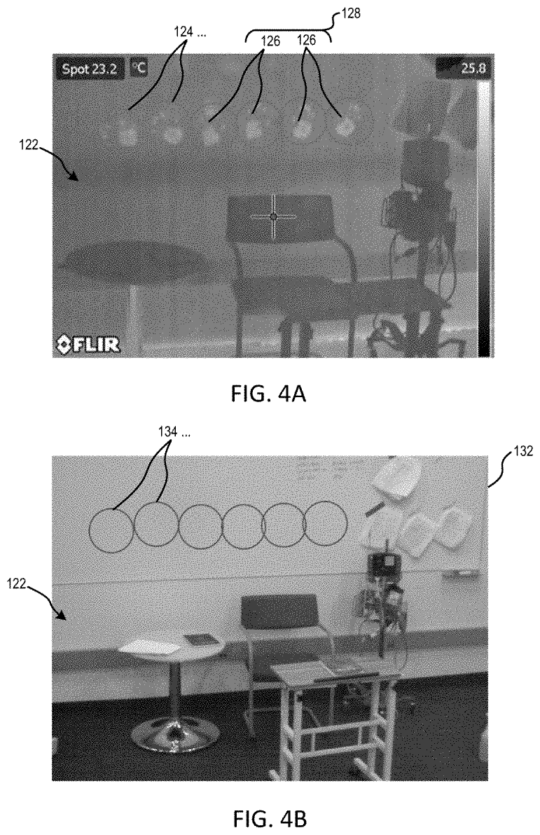

FIG. 4A illustrates the capture of multiple thermal signatures 126 on a surface 122 using the image processing system 104 of FIG. 1. Thermal signatures 126 in a patient examination area are captured by the thermal imaging device 102. Multiple handprints appear on the surface 122 (wall) behind a chair. While these handprints may not be visible to the eye, they are clearly distinguished by the thermal imaging device 102. Collectively, the thermal signatures 126 may be referred to as a heat map 128. Circles indicate touched regions 124 of the surface 122. However, the touched regions may be any shape—rectangles, ellipses, or shapes similar to the object that touched the surface 122, such as a handprint.

FIG. 4B is a visual representation 132 including graphical overlays 134 corresponding to the touched region(s) 124. The graphical overlays 134 may include various shapes, such as circles, rectangles, handprints, or the like, which are added to the visual representation 132 by the image processing system 104 to highlight the touched region(s) 124. Other forms of highlighting may be used to distinguish touched region(s) 124, such as different colors or levels of brightness.

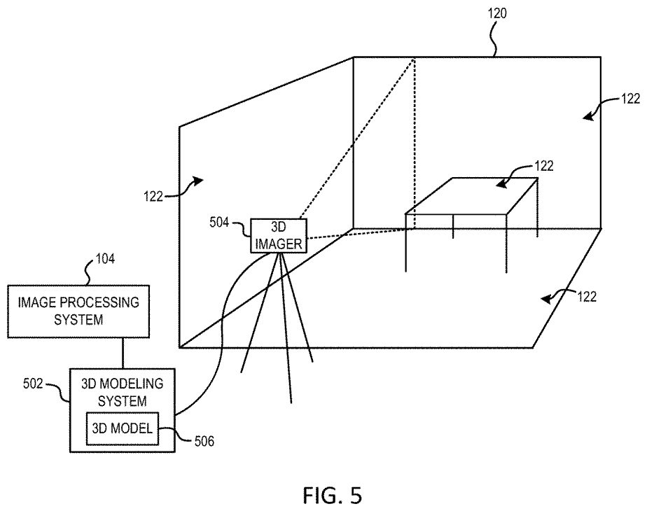

As shown in FIG. 5, the image processing system 104 may receive input from a 3D modeling system 502, which may include, or be coupled to, a 3D imager 504, such as a Light Detection and Ranging (LIDAR) camera (shown) and/or a stereographic camera. For example, a LIDAR camera may be embodied as an Intel® RealSense® L515 LIDAR camera, available from Intel Corporation of Sunnyvale, Calif., supported by the open-source Intel® RealSense® SDK 2.0.

In operation, the 3D imager 504 scans the surfaces 122 of the area of interest 120 and generates a 3D model 506. The 3D model 506 may be represented by any suitable data structure, such as a list of surfaces 122 and their orientations and spatial coordinates.

In some embodiments, the 3D model 506 may be generated manually (using CAD based on architectural drawings), with a RGBD camera (e.g., Microsoft® Kinect® or Intel® RealSense® Depth camera), using image stitching with a 2D or 3D image, or in other ways.

The image processing system 104 uses the 3D model 506 to generate the visual representation 132 of FIG. 1, as well as overlay graphical overlays 134 of the touched regions 124 upon the visual representation 132 using known image processing and graphical rendering techniques.

The touch detection system 100 may track the number of times that a particular region 124 of a surface 122 is touched. Thereafter, when generating a visual representation 132 for display on the display device 130, the touch detection system 100 may highlight or otherwise emphasize certain graphical overlays 134 of the touched regions 124 based on how many times they were touched.

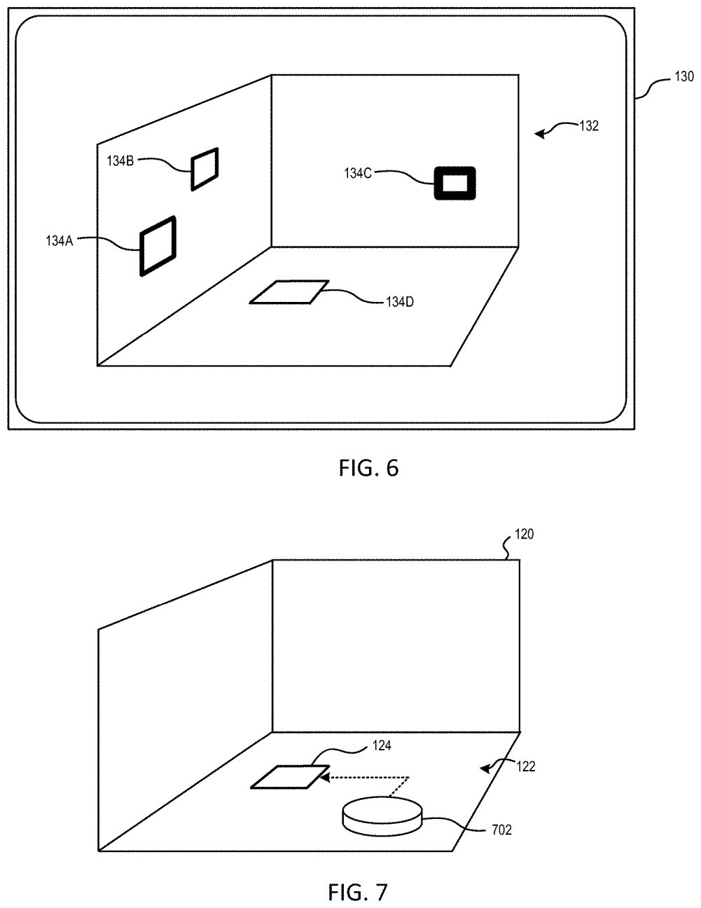

For example, as illustrated in FIG. 6, four graphical overlays 134A-134D are displayed. In one embodiment, the thickness of the lines used in the graphical overlay 134A-134D vary based on the frequency of contact. For example, graphical overlay 134D may represent a region 124 that has been touched the least, whereas graphical overlay 134C represents a region 124 has been touched the most. The graphical overlays 134A-D may be differentiated using various other techniques, including adjusting its color or brightness, adding a numerical legend, or the like. In some embodiments, a user may be able to filter the display of graphical overlays 134A-D to show only ones exceeding a particular number of contacts, allowing a cleaner to prioritize cleaning tasks or take other remedial measures, such as covering “hot spot” areas with plastic covers.

FIG. 7 illustrates another embodiment in which a cleaning robot 702 is automatically dispatched to clean a contacted region 124 of a surface 122 based on the heat map used in the generation of the visual representation 132 of FIG. 6. For example, the cleaning robot 702 may include a Braava® robot mop available from iRobot Corporation of Bedford, Mass. A wide variety of robots capable of navigating floors and/or walls may be used, including flying drones and/or cleaning systems that are integrated into the environment (e.g., sprayers, wipers, etc.), all of which are contemplated by the term, “cleaning robot.”

In some embodiments, the cleaning robot 702 may spray disinfectant on the contacted region 124 and/or sterilize the contacted region 124 with ultraviolet light. Techniques for navigating a cleaning robot 702 to a destination in two- or three-dimensional space are known in the art and may rely on the 3D model 506 of FIG. 5. Various routing algorithms and object-avoidance techniques may be used to maximize efficiency and ensure safety in a crowded environment.

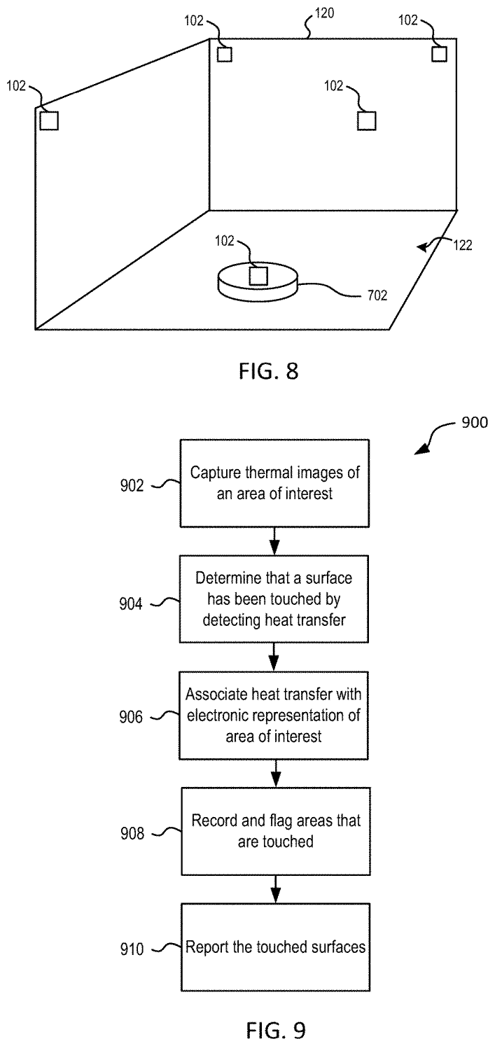

In some embodiments, as shown in FIG. 8, the touch detection system 100 includes a plurality of thermal imaging devices 102 (and, optionally, visible light cameras 108) configured to work in concert with one another and with the image processing system 104 of FIG. 1. The thermal imaging devices 102 can be arranged to encompass a large proportion of an area, up to a complete visual coverage of the area of interest 120. The image processing system 104 can be accordingly configured to fuse thermal images, distinguish redundant/overlapping areas, and report only once the touched surfaces 122 or regions thereof that may appear within the field of view of multiple thermal imaging devices 102.

Optionally, thermal images of the area of interest 120 may be captured while a cleaning step is underway. The cleaning robot 702, itself, may include a thermal imaging device 102. Thermal images of the surfaces 122 touched during cleaning can then be compared to those touched earlier. In these cases, the cleaning robot 702 is in electronic communication with the touch detection system 100 for identifying, and navigating to, areas to be cleaned.

Referring to FIG. 9, another aspect of the present disclosure provides a method 900 of determining surfaces that have been touched. The method 900, which can be accomplished using the touch detection system 100 described above, may begin with the capture 902 of thermal images in an area of interest by a thermal imaging device. When a subject (e.g., a human) touches a surface within the area of interest, an image processing system determines 904 that the surface has been touched by detecting transient heat that is transferred to or from the surface due to a differential between the body warmth of the subject and the temperature of the surface. The transferred heat is associated 906 by the image processing system with a corresponding electronic representation of the area of interest (e.g., a photograph, diagram, or 2D of 3D schematic image of the area). The system can record 908 areas that are touched and flag these areas, as well as track the number of times an area has been touched. Finally, the image processing system may report 910 the touched surfaces. The user receiving the report can then take a prescribed action, such as executing a targeted cleaning regime.

The disclosure herein provides several advantages over current approaches to cleaning in a variety of applications by providing real-time touch tracking in a clinical setting. For example, it can improve the turnover of important clinical units such as emergency triage rooms by decreasing the amount of cleaning time. An intuitive graphical user interface ensures that the appropriate information is transmitted in a clear format. It can also be used to monitor how well current cleaning procedures provide coverage of the high-touch hotspots. Further, thermal monitoring does not pose a risk to patient confidentiality, as faces are not recognizable from thermal images.

It is to be understood that the systems described herein can be implemented in hardware, software, firmware, or combinations of hardware, software and/or firmware. In some examples, image processing may be implemented using a non-transitory computer readable medium storing computer executable instructions that when executed by one or more processors of a computer cause the computer to perform operations. Computer readable media suitable for implementing the control systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements an image processing system described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of identifying a region of a surface that has been contacted by a subject, comprising:
monitoring, using a thermal imaging device, an area of interest comprising the surface;
detecting, using an image processing system, heat that is transferred to or from the region of the surface after being contacted by the subject; and
storing an indication of the contacted region of the surface.

2. The method of claim 1, further comprising:
reporting the contacted region.

3. The method of claim 2, wherein reporting the contacted region comprises creating a visual representation of the area of interest upon which a graphical overlay corresponding to the contacted region is displayed.

4. The method of claim 3, wherein creating a visual representation comprises a creating a virtual or augmented reality image.

5. The method of claim 1, further comprising:
directing a cleaner to the contacted region.

6. The method of claim 5, wherein directing comprises:
navigating a cleaning robot to the contacted region.

7. The method of claim 1, further comprising:
tracking a number of times that one or more regions of at least one surface of the area of interest have been contacted; and
reporting at least one of the one or more regions that have been contacted most frequently for more intensive or frequent cleaning.

8. The method of claim 1, wherein detecting comprises:
detecting, using the image processing system, a change in temperature of the region of the surface;
determining whether the change in temperature is consistent with human contact with the region of the surface; and
if the change in temperature is consistent with a human contact, identifying the region as a contacted region.

9. The method of claim 8, wherein determining whether the change in temperature is consistent with human contact comprises evaluating the change temperature based on at least one of a magnitude or rate of change in temperature of the region of the surface.

10. The method of claim 1, further comprising:
prior to monitoring, generating a three-dimensional representation of one or more surfaces in the area of interest.

11. The method of claim 10, wherein the three-dimensional representation of the area is captured by a three-dimensional imager.

12. A touch detection system, comprising:
a thermal imaging device configured to capture an image of an area of interest that has at least one surface;
an image processing system configured to detect heat transferred to or from a region of the at least one surface after the region has been contacted by a subject; and
a storage device configured to store an indication of the contacted region of the surface.

13. The touch detection system of claim 12, further comprising:
a communication interface configured to report the contacted region.

14. The touch detection system of claim 12, further comprising:
a camera configured to obtain a visual representation of the area of interest; and
a display device configured to display a graphical overlay corresponding to the contacted region on the visual representation.

15. The touch detection system of claim 14, wherein the visual representation comprises a virtual or augmented reality image.

16. The touch detection system of claim 13, wherein the communication interface is configured to direct a cleaner to the contacted region.

17. The touch detection system of claim 16, wherein the cleaner comprising a cleaning robot.

18. The touch detection system of claim 12, further comprising:
a processor configured to track a number of times that one or more regions of at least one surface of the area of interest have been contacted; and
a communication interface configured to report at least one of the one or more regions that have been contacted most frequently for more intensive or frequent cleaning.

19. The touch detection system of claim 12, wherein the image processing system is configured to:
detect a change in temperature of the region of the surface;
determine whether the change in temperature is consistent with human contact with the region of the surface; and
if the change in temperature is consistent with a human contact, identify the region as a contacted region.

20. The touch detection system of claim 19, wherein the image processing system is configured to determine whether the change in temperature is consistent with human contact by evaluating the change temperature based on at least one of a magnitude or rate of change of change in temperature of the region of the surface.

21. The touch detection system of claim 12, further comprising:
a three-dimensional modeling system configured to generate a three-dimensional model of one or more surfaces in the area of interest.

22. A non-transitory computer-readable medium comprising program code that, when executed by a processor, causes the processor to perform a method of identifying a region of a surface that has been contacted by a subject, the method comprising:
monitoring, using a thermal imaging device, an area of interest comprising the surface;
detecting, using an image processing system, heat that is transferred to or from the region of the surface after being contacted by the subject; and
storing an indication of the contacted region of the surface.
